(19) United States
(12) Patent Application Publication
(10) Pub. No.: US 2001/0016954 A1
(43) Pub. Date: Aug. 23, 2001

(54) ROOT SPECIFIC PROMOTERS

(76) Inventors: HOWARD JOHN ATKINSON, NORTH YORKSHIRE (GB); CATHERINE JANE LILLEY, BRADFORD (GB)

Correspondence Address: DAVID A JACKSON, KLAUBER & JACKSON, 411 HACKENSACK AVENUE, HACKENSACK, NJ, 07601

(21) Appl. No.: 09/077,595

(22) PCT Filed: Nov. 28, 1996
(86) PCT No.: PCT/GB96/02942

§ 371 Date: None

(30) Foreign Application Priority Data
Nov. 29, 1995 (GB) 9524395.2

(51) Int. Cl. A01H 5/00

(52) U.S. Cl. 800/287

(57) ABSTRACT

This invention relates to the control of pests. In particular the invention relates to the protection of plants against parasitic nematodes.

The invention provides nucleic acid comprising a transcription initiation region capable of directing expression predominantly in the roots of a plant, and a sequence which encodes an anti-nematode protein.

This invention relates to the control of pests. In particular the invention relates to the protection of plants against parasitic nematodes.

Nematodes cause global crop losses that have been valued at over $100 billion per year. Examples of particularly important species include Meloidogyne incognita and M. javanica (a wide range of crops), Globodera spp (potato cyst nematodes) Heterodera schachtii (beet cyst nematode) and Heterodera glycines (soybean cyst-nematode). In addition to direct feeding damage, some nematodes are involved in disease associations. In particular, the Dorylaimid nematodes, (Trichodorus, Paratrichodorus, Longidorus, Paralongidorus and Xiphinema) transmit NEPO and TOBRA viruses.

The majority of plant parasitic nematodes attack plant roots rather than aerial tissues. Examples of root parasitic nematodes are species of the genera Heterodera, Globodera, Meloidogyne, Hoplolaimus, Helicotylenchus, Rotylenchoides, Belonolaimus, Paratylenchus, Paratylenchoides, Radopholus, Hirschmanniella, Naccobus, Rotylenchulus, Tylenchulus, Hemicycliophora, Criconemoides, Criconemella, Paratylenchus, Trichodorus, Paratrichodorus, Longidorus, Paralongidorus, Rhadinaphelenchus, Tylenchorhynchus, Hemicriconemoides, Scutellonema, Dolichodorus, Gracilacus, Cacopaurus, Xiphinema and Thecavermiculatus. Host ranges of these species include many of the world's crops and are defined elsewhere (Luc et al, Plant Parasitic Nematodes in Subtropical and Tropical Agriculture, CAB International, Wallingford, p629 (1990), Evans et al, Plant Parasitic Neematodes in Subtropical and Tropical Agriculture, CAB International, Wallingford, p648 (1993)).

Root-parasitising nematodes may be ecto- or endo-parasites. In many examples the mouth stylet is inserted and cell contents are removed. Several economically important groups of root parasites have females with a prolonged sedentary phase during which they modify plant cells into nematode feeding sites. Nematodes are the principal animal parasites of plants. They are not herbivores in that they do not ingest whole cells and plant cell walls as characterises the feeding of herbivores such as many insects, molluscs and mammals. The different host-parasite relationships of root feeding nematodes are summarised by Sijmons et al (Annual Review of Phytopathology 32: 235-59 (1994)). The requirements for control are therefore distinct from those of other pests such as insects.

This invention has application to any transformable or potentially transformable crop whose root system is damaged by nematodes. This includes a wide range of temperate and tropical crops. The temperate crops to which root parasitic nematodes cause economic damage include: potato, sugar beet, vegetables, oil seed crops, grain, legumes, cereals, grasses, forage crops, forest trees, fruit trees, nut trees, soft fruits, vines, ornamental and bulb crops. Information on the nematode genera and species damaging each of these is given in Evans (1993, supra).

A wide range of crops also suffer economic loss from nematodes in tropical and subtropical agriculture. These include: rice (growing in all its cropping ecosystems), cereals, root and fibre crops, food legumes, vegetables, peanut, citrus, fruit trees, coconut and other palms, coffee, tea and cocoa, bananas, plantains, abaca, sugar cane, tobacco, pineapple, cotton, other tropical fibre crops, and spices. Details of the economic genera and the damage they cause are provided by Luc et al (1990, supra).

Control of nematodes currently relies on three principal approaches, chemicals, cultural practices and resistant varieties, often used in an integrated manner (Hague and Gowen, Principles and Practice of Nematode Control in Crops (Brown, R. H. and Kerry, B. R., eds.), pp. 131-178, Academic Press (1987)). Chemical control is not only costly in the developing world but involves application of compounds including carbamates, such as Aldicarb, which is one of the most toxic and environmentally hazardous pesticides in widespread use. Toxicological problems and environmental damage caused by nematicides has resulted in either their withdrawal or severely restricted their use. They are the most toxicologically and environmentally unacceptable pesticides in widespread use posing considerable risk to aquatic ecosystems and drinking water supplies (Gustafson, D I Pesticides in Drinking Water, N. Carolina, USA, p241 (1993)).

Cultural practices such as crop rotation are widely used but they are rarely adequate alone. Resistant cultivars have been a commercial success for a limited range of crops but the approach is unable to control many nematode problems for a variety of reasons (Roberts, Journal of Nematology, 24:213-227 (1992)).

Resistance of crops to nematodes is clearly an important goal. For nematodes, resistance is defined by the success or failure of reproduction on a genotype of a host plant species. Dominant, partially dominant and recessive modes of inheritance occur based on one or more plant genes. A gene-for-gene hypothesis has been proposed in some cases with typically a dominant R-gene for resistance being countered by a recessive V-gene for virulence in the nematode. Two examples of resistance introduced by breeders are as follows.

In relation to Globodera spp, different sources of resistance occur and allow subdivision of potato cyst nematode populations in Europe into two species, each with a number of pathotypes. The European pathotyping scheme envisages eight pathotypes, but the validity and utility of some of the distinctions it makes have been challenged (Trudgill, 1991 Annual Review of Phytopathology 29: 167-192). Pathotypes are defined as forms of one species that differ in reproductive success on defined host plants known to express genes for resistance. Use of resistant cultivars may favour selection of certain pathotypes and also favour species unaffected by effective resistance against other nematodes. The H1 gene conferring resistance to certain pathotypes of Globodera rostochiensis provided virtually qualitative resistance against UK populations of this nematode, and is widely used commercially. Within the UK, cv Maris Piper expresses H1 and is a highly successful resistant cultivar. Unfortunately, its widespread use in Britain is correlated with an increased prevalence nationally of G. pallida to which it is fully susceptible.

A second example occurs in relation to Meloidogyne spp., morphologically similar forms or races occur with differential abilities to reproduce on host species. The standard test plants are tobacco (cv NC95) and cotton (cv Deltapine) for the four races of M. incognita whereas the two races of M. arenaria are differentiated by peanut. (cv Florrunner). The single dominant gene in tobacco cv NC95 confers resistance to M. incognita races 1 and 3 but its cropping in the USA has increased the prevalence of other root-knot nematodes particularly M. arenaria. Most sources of resistance are not effective against more than one species of root-knot nematode with the notable exception of the LMi gene from Lycopersicum peruvanium which confers resistance to many species except M. hapla. Another limitation of resistance genes identified in tomato, bean and sweet potato is a temperature dependence which renders them ineffective where soil temperature exceeds 28° C.

The limitations of conventional control procedures provide an important opportunity for plant biotechnology to produce effective and durable forms of nematode control. Principal advantages are

(i) an approach to pest control that does not require other changes to agronomic practices;

(ii) a reduction in toxicological and environmental risks associated with chemical control; and

(iii) the provision of. effective, appropriate and inexpensive crop protection.

Designs for such novel plant defences can be envisaged that lack environmental, producer or consumer risk while providing substantial economic benefits for both the developed and developing world.

Plant defences against nematodes are known that are additional to the specific genes for resistance reviewed by Roberts ((1992) supra). Pre-formed plant defensive compounds may be particularly effective against initial events such as invasion and feeding by nematodes. Such compounds may be lethal to nematodes or act as semiochemicals causing premature exit from the plant. The secondary metabolites involved have been considered by Huang (An advanced treatise on Meloidogyne volume 1 Biology & Control, p 165-174, J. N. Sasser & C. C. Carter (eds), North Carolina. State University graphics(1985) although none of these are proteins.

Proteins with roles in plant defence are divided by Bowles (Ann. Rev. of Biochem., 59:873-907 (1990)) into three groups:

i) those that directly change the properties of the extracellular matrix;

(ii) proteins that have a known direct biological activity against the pathogen or catalyse the synthesis of antimicrobial products; and

iii) proteins whose appearance can be correlated with defence response but which are of unknown function.

Nematode interactions with roots can result in changes in expression of these classes. For instance, changes in peroxidases occur (group (i) above) (Zacheo, G. and Bleve-Zacheo, T., Pathogenesis and Host Specificity in Plant Diseases, Vol II Eukaryotes, ed. Kohmoto, K. Singh U. S. and Singh, R. P., Elsevier, Oxford, UK, p.407 (1995)). Hammond-Kosack et al (Physiol. Mol. Plant Pathol., 35: 495-506 (1989)) showed that pathogenesis-related proteins are induced in plant leaves when nematodes invade roots (group (ii) above) and the promoter of Wun-1 responds to cyst nematode invasion of roots (group (iii) above) (Atkinson et al, Trends in Biotechnology 13: 369-374 (1995)). Changes in gene expression within roots are considered in detail by Sijmons et al (1994) and Atkinson et al (supra).

One of the most basic requirements for engineered resistance against a nematode is a plant transformed with an element (promoter) regulating expression of a coding region for an effector protein that disrupts some aspect of the parasitism. Two principal strategies have been devised to-date for nematode control based on transgenic plants utilising two distinct classes of effectors.

The first approach (type 1) is centred on expressing in plants, proteins that do not impair plant growth and yields, but do have anti-nematode effects. This is the approach relevant to this application. The best characterised to-date are proteinase inhibitors.

An example of such an approach can be found in EP-A-0502730 which discloses the use of proteinase inhibitors, eg cowpea trypsin inhibitor (CpTi) and oryzacystatin, to protect plants from nematode parasitism and reproduction. Transgenic plants which express nucleic acid coding for such proteinase inhibitors are also disclosed. Such transgenic plants will therefore be nematode resistant.

These are natural, defence-related, proteins induced in aerial parts of plants and certain other tissues by wounding and herbivory. While they are induced systemically in the aerial parts of plants by nematode parasitism of roots they are surprisingly not present in roots. Cowpea trypsin inhibitor has some potential against insects when expressed as a transgene (Hilder et al, Nature 220: 160-163 (1987)). For those advocating their use in transformed plants, PIs have the particular advantage of already being consumed by humans in many plant foods.

The second approach to nematode control (type 2) is not relevant to the present application. It is based on indirect control of nematodes by preventing stable feeding relationships using a concept that has analogy with the plant cell suicide concept of engineered emasculation in maize. This involves expression of a plant cell lethal sequence under the control of a tapetal cell-specific promoter and destroys the male flower (Mariani et al, Nature 347: 737-741 (1990)). This approach has been applied to control of cyst and root-knot nematodes (Gurr et al, Mol. Gen. Genet. 226: 361-366 (1991); Opperman et al (1994)). It relies on identification of feeding site specific promoters or other bases for limiting plant cell death to the feeding cell of the parasite (see Atkinson et al (1995) supra. It is the search for such promoters that has underpinned much of the work on nematode-responsive plant genes.

There is a clear distinction between direct control of the nematode with anti-parasitic proteins and indirect control by impairing specific plant cells on which certain nematodes depend. These two strategies require very different promoters to provide expression patterns in plants of interest.

The approach taken in this application has been to identify promoters of value for a generic defence against a wide range of nematode genera. This is important because many important genera attacking plants such as Belonolaimus, Helicotylenchus, Hirschmanniella, Paratylenchus, Radopholus, Xiphinema, Trichodorus, Paratrichodorus, Longidorus, Paralongidorus, Criconemella, Rhadinaphelenchus, Tylenchorhynchus, Hemicycliophora, Hemicriconemoides, Hoplolaimus, Scutellonema, Aorolaimus, Dolichodorus, Rotylenchus, Hemicriconemoides, Paratylenchus, Gracilacus and Cacopaurus do not induce feeding cells. The need is to define genes that are known to be differentially expressed in roots with little expression elsewhere in the plant and to use the promoters associated with these genes. Such promoters enable the provision of pre-formed defences that have no relationship with any known plant defence against nematodes.

Thus, in a first aspect, the present invention provides nucleic acid comprising a transcription initiation region capable of directing expression predominantly in the roots of a plant, and a sequence which encodes an anti-nematode protein.

Suitably, the transcription initiation region will be a promoter, but the invention also encompasses nucleic acid which comprises only those parts or elements of a promoter required to initiate and control expression. Generally, the nucleic acid of the invention will also include a transcription termination region.

The transcription initiation region can be one which is unresponsive to nematode infection. Alternatively, it can be one which will drive expression throughout the roots of a plant in the absence of any nematode infection, but which exhibits a degree of “up-regulation” at an infected locale once infection of the plant occurs.

In the context of the present application, the term “anti-nematode protein” will include all proteins that have a direct effect on nematodes. Examples of such proteins include collagenases (Hausted et al, Conference on Molecular Biology of Plant Growth and Development, Tucson, Ariz. (1991)) and lectins (see, for example, WO 92/15690 which showed that a pea lectin delayed development of G. pallida to some extent when expressed transgenically). Cholesterol oxidase expression in transgenic tobacco plants caused the death of bollweevil larvae (Purcell et al., Biochem. Biophys. Res. Comm. 196: 1406-1413, (1993)) and may also be effective against nematodes. Expression of peroxidase or oxidase in plants may defend them against nematodes to which it is lethal (Southey Laboratory methods for work with plant and soil nematodes Ministry of Agriculture, Fisheries and Food, Reference Book 402 HMSO 202pp 1986). Transgenic potato plants expressing the hydrogen peroxidase-generating enzyme glucose oxidase have enhanced resistance to bacterial and fungal pathogens (Wu et al., Plant Cell, 7:1357-1368, 1995). It is also known that reduced peroxidase activity in tomato plants is associated with increased susceptibility to Meloidogyne incognita (Zacheo et al., Physiological & Molecular Plant Pathology, 46:491-507 (1995)).

Expression of antibodies in plants (Hiatt et al, Nature 342: 76-78 (1989); Schots et al, Netherlands Journal of Plant Pathology 98: 183-191 (1992) may also provide anti-nematode proteins of interest. Antibodies of potential interest include those raised against nematodes (Atkinson et al, Annals of Applied Biology 112: 459-469 (1988) and single chain antibody fragments when used alone or when conjugated to an appropriate toxin (Winter and Milstein, Nature 349: 293-299 (1993). This example has been demonstrated by the expression in plants of antibodies directed against a fungal cutinase (Van Engelen et al., Plant Molecular Biology 26: 1701-1710 (1994)). A toxin of interest alone or conjugated to an antibody can include any toxin of Bacillus thuringiensis that is effective against nematodes. One report to date is for the efficacy of an exotoxin only (Devidas and Rchberger, Plant Soil 145: 115-120 (1992).

The term anti-nematode protein also includes, but is not restricted to, proteinase inhibitors against all four classes of proteinases and all members within them (Barrett, A. J., Protein Degradation in Health and Disease, Ciba Foundation Syi,posium 75: 1-13 (1980)).

Other examples of “anti-nematode proteins” include any protein inhibitor of a nematode digestive enzyme. Plant parasitic nematodes contain several enzymes including proteinases, amylases, glycosidases and cellulases (Lee, The Physiology of Nematodes Oliver & Boyd pp153 (1965)). Starch depletion occurs in nematode feeding cells and has been attributed to nematode amylase activity (Owens & Novotny, Phytopathology, 50:650, 1960). α-amylase inhibitors expressed in transgenic plants provide resistance to pea weevil larvae (Schroeder et al., Plant Physiology, 107:1233-1239: (1995)) and bruchid beetles (Shade et al., Bio/Technology, 12:793-796: (1994)).

In general the protein will be one which may have a biological effect on other organisms but preferably has no substantial effect on plants.

In one embodiment of this aspect of the invention, the transcription initiation region includes or is the promoter from the b1-tubulin gene of Arabidopsis (TUB-1). Northern blots have shown that the transcript of this gene accumulates predominantly in roots, with low levels of transcription in flowers and barely detectable levels of transcript in leaves (Oppenheimer et al, Gene, 63:87-102 (1988)). In another embodiment the transcription initiation region is the promoter from the metallothionein-like gene from Pisum sativum (PsMT_{A}) (Evans et al, FEBS Letters, 262:29-32 (1990)). The PsMT_{A} transcript is abundant in roots with less abundant expression elsewhere.

Further embodiments of this aspect of the invention include the transcription initiation regions comprising, or being the RPL16A promoter from Arabidopsis thaliana (the RPL16A gene from A. thaliana encodes the ribosomal protein, L16, its expression. being cell specific) or the ARSK1 promoter from A. thaliana (the ARSK1 gene encodes a protein with structural similarities to seine/threonine kinases and is root specific). These two promoters are described in more detail in Examples 6 and 7 and the preceding paragraph thereto. Further embodiments include the promoter of the A. thaliana AKTI gene. This gene encodes a putative inwardly-directed potassium channel. The promoter preferentially directs GUS expression in the peripheral cell layers of mature roots (Basset et al., Plant Molecular Biology, 29: 947-958 (1995) and Lagarde et al., The Plant Journal, 9: 195-203 (1996). Also included is the promoter of the Lotus japonicus LJAS2 gene, a gene encoding a root specific asparagine synthetase. Expression of the gene is root specific, as judged by nothern blot analysis (Waterhouse et al., Plant Molecular Biology, 30: 883-897 (1996).

The present invention also describes, as a separate aspect, the manipulation of a transcription initiation region, especially a promoter, to increase its usefulness. Such manipulation may be used to develop a root-specific promoter. In particular, promoter deletions may be created to identify regions of the promoter which are essential. or useful for expression in roots and/or to manipulate a promoter to have greater root specificity. Such promoters may be used in conjunction with, but are not limited to, the other aspects of the invention herein described, specifically for use in predominant expression of an anti-nematode protein in the roots of a plant.

A suitable promoter (PsMT_{A}) manipulated as described above is detailed below and in the Examples. The specificity of the promoter is altered by creating deleted versions (constructs) of the promoter. The deleted versions have altered promoter activity and can be used to describe embodiments of the invention. As will be understood by the person skilled in the art, the technique of manipulation can be applied to any transcription initiation region.

As will be understood by the skilled person, any transcription initiation region which directs expression of a gene(s) predominantly in the roots of a plant can be used according to the invention.

Promoter tagging has been achieved through random T-DNA-mediated insertion of a promoterless gusA gene (Lindsey et al, Transgenic Res. 2: 33-47 (1993); Topping et al, Development 112: 1009-1019 (1991). This provides transgenic β-glucuronidase activity as a reporter gene that is calorimetrically detectable in plants (Jefferson et al, EMPO J. 227: 1229-1231 (1987). Screening transformed plants e.g. Arabidopsis, allows the identification of any promoter tagged by insertion of the gusA gene that provides root-specific expression. This approach has been applied to identify differential gene expression in nematode-induced feeding structures (Goddijn et al (1993) , Sijmons et al (1994) supra, and patent application Ser. No. PCT/EP92/02559).

It follows that similar approaches can be used to ensure no down-regulation occurs for a root-specific gene on infection of the transformed plant by nematodes as described in this invention. Once such a promoter is tagged, those practised in the art will be familiar with the techniques of inverse Polymerase Chain Reaction (inverse PCR; Doses et al, Plant Molecular Biology 17: 151-153 (1991) which will isolate the region 5′ to the inserted promoter. If necessary, this provides a clone for screening a genomic library of the plant species (e.g. Arabidopsis) to identify putative promoter regions. Methodology for library screening is given in Sambrook et al, infra (1989). Insertion of gusA under control of the putative promoter into a plant such as Arabidopsis provides a positive basis for confirming patterns of reporter (GUS) activity. Confirmation is achieved if the root-specific, expression occurs in uninfected roots as in the original tagged line. This pattern of expression should not be down-regulated by nematode infection as occurs for several promoters examined to date.

The skilled person will appreciate that it is not a requirement of the present invention based on a type I defence that no expression occurs outside of the root system. Providing expression is predominantly in the root system of healthy roots the nucleic acid of the invention offers the prospect of a preformed defence that is not dependent on a response to nematode invasion of the roots.

In addition, promoter deletion studies (Opperman et al, Science, 263:221-223 (1994)) have established that the spatial pattern of expression provided by a promoter can be modified. Therefore unwanted, minor spatial patterns of expression can be eliminated by modification of promoters so that only the pattern of interest remains. Thus, this will allow the possibility of eliminating aerial expression without loss of root expression.

The skilled person will appreciate that identification of suitable transcription initiation regions will be relatively straightforward and can be carried out using techniques well known in the art.

The nucleic acid of the invention can be in the form of a vector. The vector may for example be a plasmid, cosmid or phage. Vectors will frequently include one or more selectable markers to enable selection of cells transfected (or transformed: the terms are used interchangeably in this specification) with them and, preferably, to enable selection of cells harbouring vectors incorporating heterologous DNA. Vectors not including regulatory sequences are useful as cloning vectors.

Nucleic acid of the invention, eg DNA, can be prepared by any convenient method involving coupling together successive nucleotides, and/or ligating oligo- and/or poly-nucleotides, including in vitro processes, but recombinant DNA technology forms the method of choice.

In a second aspect, the present invention provides the use of nucleic acid comprising a transcription initiation region capable of directing expression predominantly in the roots of a plant, in the preparation of a nucleic acid construct adapted to express an anti-nematode protein.

In a third aspect, the present invention provides a method of conferring nematode resistance on a plant which comprises the step of transforming the plant with nucleic acid as defined herein.

In a fourth aspect, the present invention provides the use of nucleic acid as defined herein in the preparation of a transgenic plant having nematode resistance.

In a fifth aspect, the present invention provides a plant cell transformed with nucleic acid as defined herein.

In a sixth aspect the present invention provides a plant comprising cells transformed with nucleic acid as defined herein.

The present invention thus provides a novel and advantageous approach to the problem of protecting plants, especially commercially important ones, from nematode infestation. In particular, the invention has the following advantages:

a) In contrast to a constitutive promoter such as CaMV35S the anti-nematode protein is expressed principally in roots and not at high levels in the yield or aerial parts of the plant;

b) This restricted expression offers advantages in overcoming regulatory or environmental criticisms of expression of anti-nematode proteins in aerial parts of plants;

c) The approach has the considerable advantage of defending any plant against more than one nematode species during concurrent or sequential parasitism at one site and for localities with dissimilar nematode problems. For example, protection could be provided for upland rice and maize against infection with Meloidogyne spp and Paratylenchus spp.

d) The potential in the previous point extends to control of two nematodes forming distinct feeding cells on one host such as Meloidogyne spp and H. glycines on soybean, Meloidogyne spp and Globodera spp on potato and Meloidogyne, Rotylenchulus on cotton.

e) A general defence against nematodes has commercial value in eliminating the need to determine the presence of nematodes or to quantify economic species.

f) It offers the plant breeding industry a nematode defence readily introduced to any transformable crop species without extensive modification for different nematodes or plant species.

Thus, the skilled person will appreciate that the present invention provides an effective and generic strategy for preventing nematode infestation.

Preferred features of each aspect of the invention are as for each other aspect, mutatis mutandis.

The invention will now be described with reference to the following examples, which should not be construed as in any way limiting the invention.

The examples refer to the figures, in which:

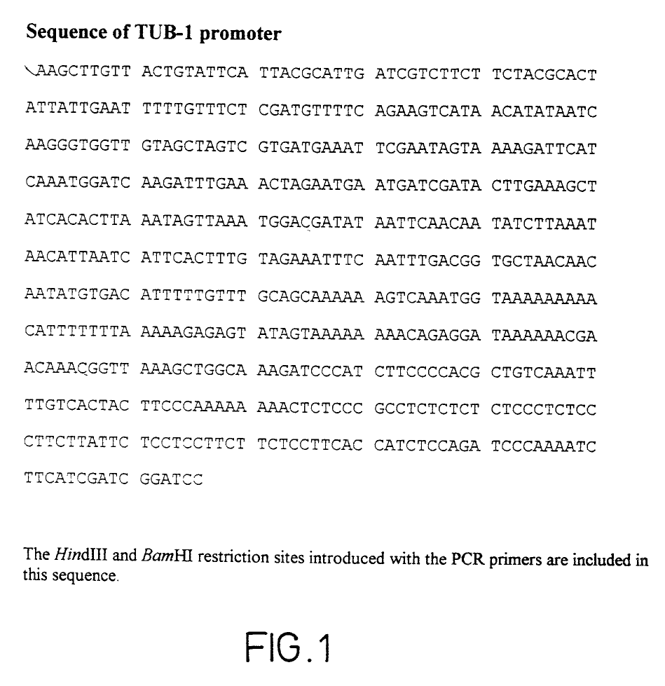

FIG. 1: shows the sequence of the TUB-1 promoter;

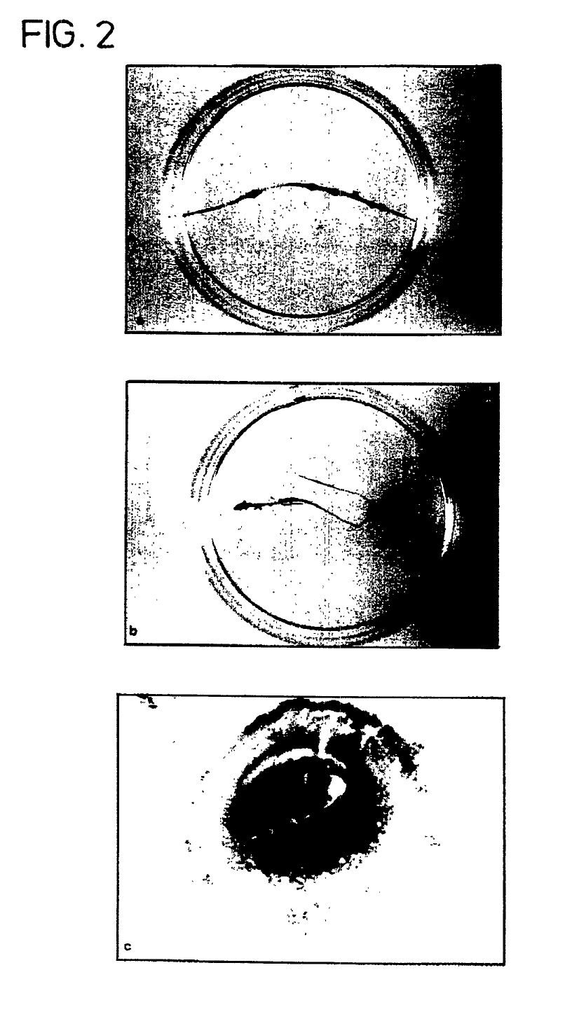

FIG. 2: shows the results of expression of GUS under the control of the TUB-1 promoter in transgenic hairy roots of tomato;

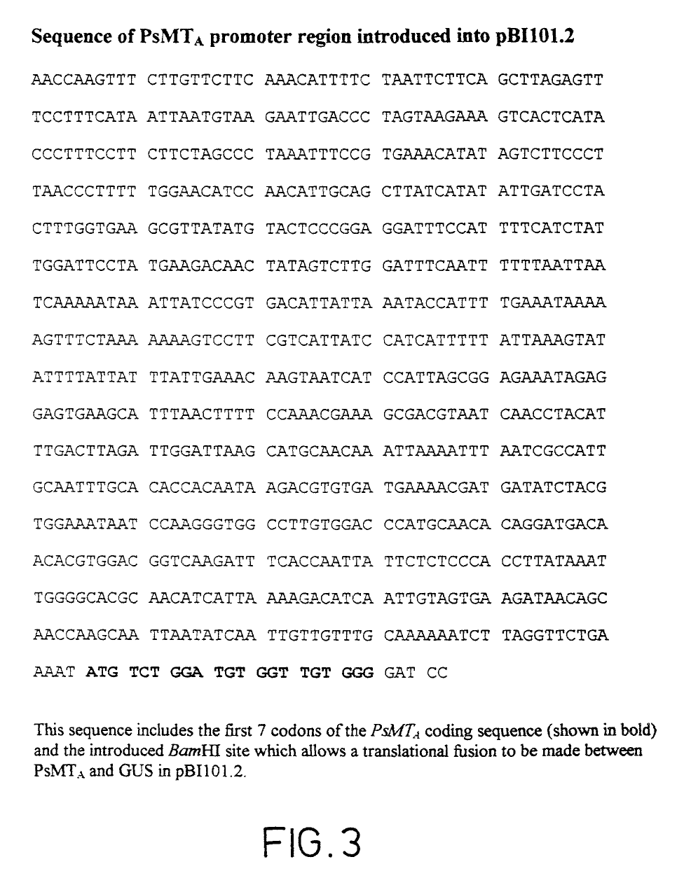

FIG. 3: shows the sequence of the PsMT_{A} promoter;

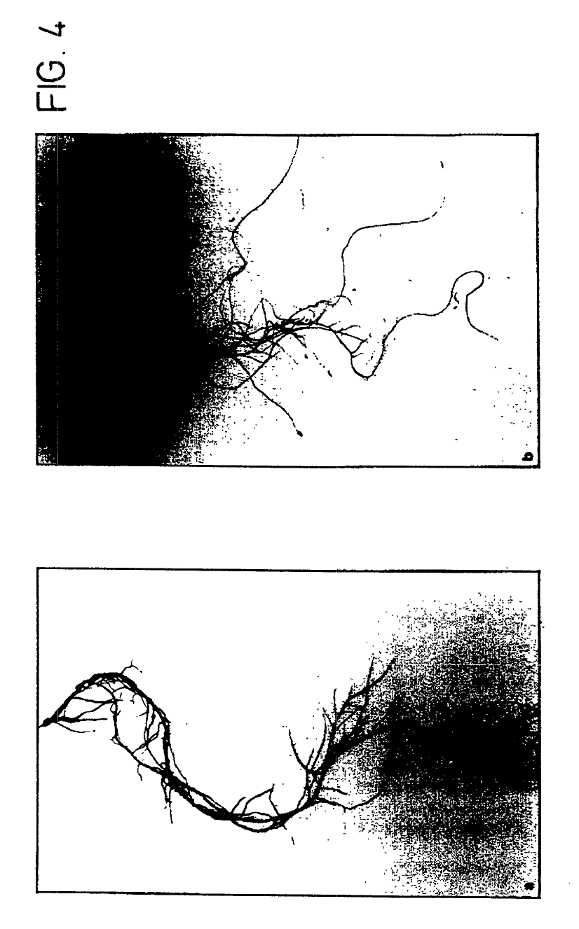

FIG. 4: shows the results of transgenic Arabidopsis roots expressing GUS under the control of the PsMT_{A} promoter;

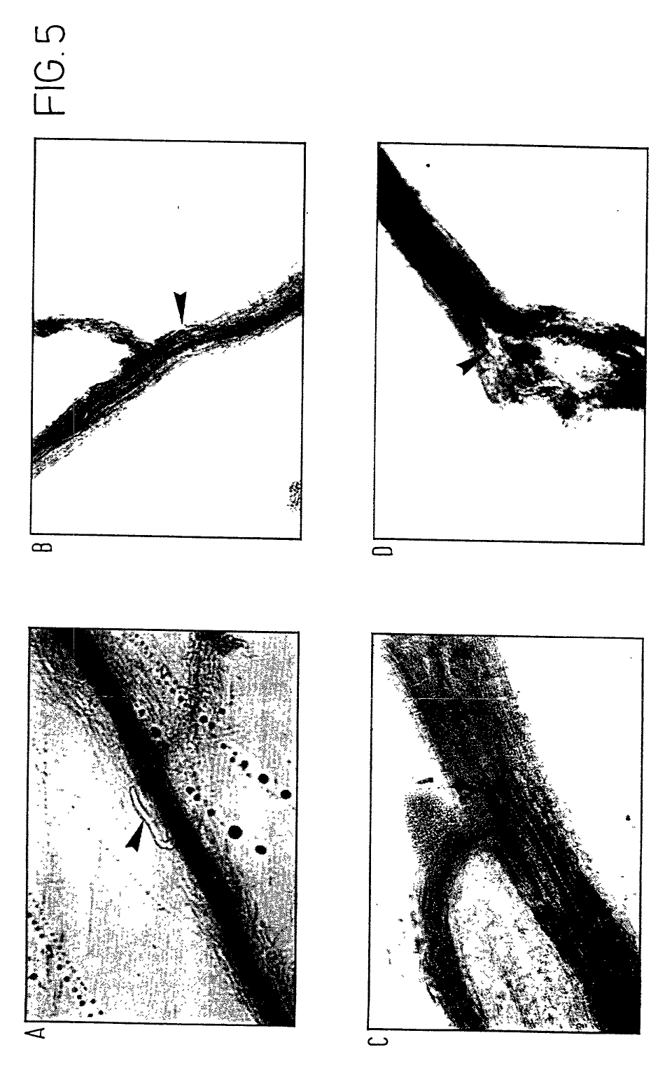

FIG. 5: shows the results of A. thaliana transformed with PsMT_{A}: GUS construct and infected with Heterodera schachtii;

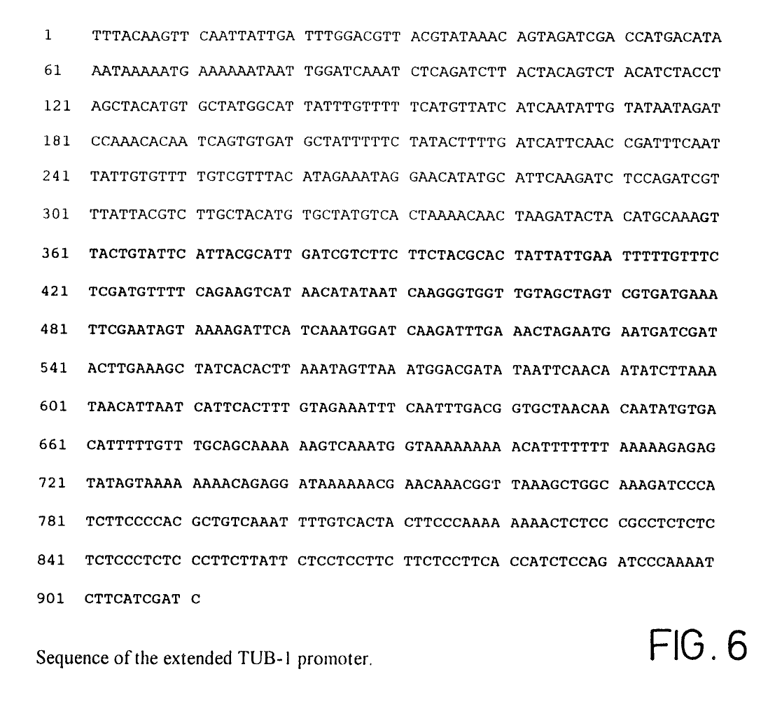

FIG. 6: shows the extended sequence of the TUB-1 promoter;

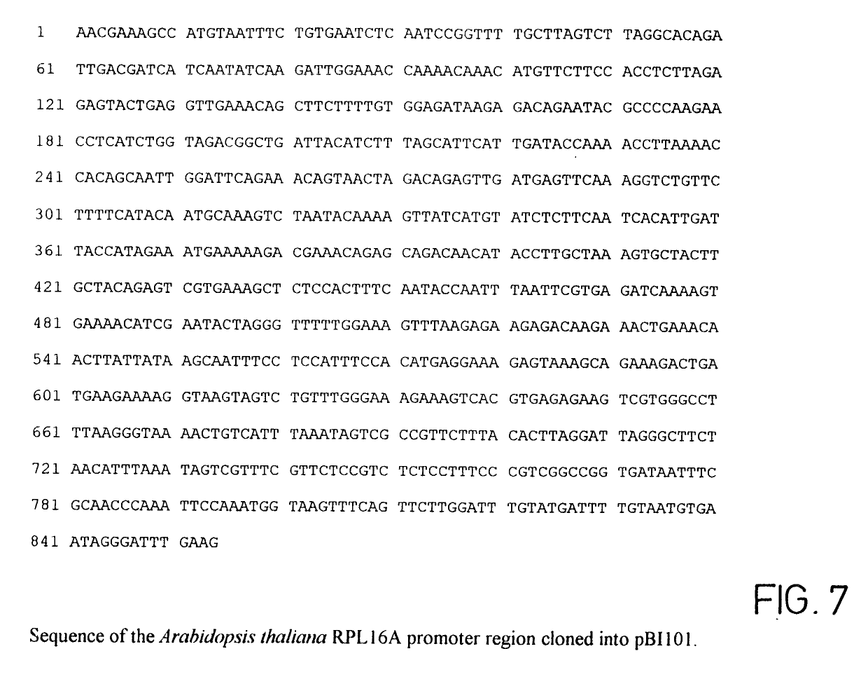

FIG. 7: shows the sequence of the A. thaliana RPL16A promoter region cloned into pBI101, in Example 6;

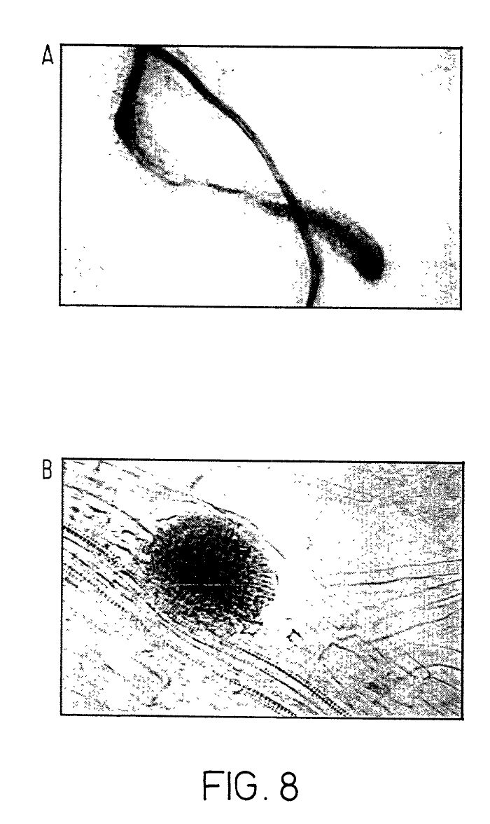

FIG. 8: shows the results of A. thaliana transformed with the RPL16A: GUS construct and stained for GUS activity;

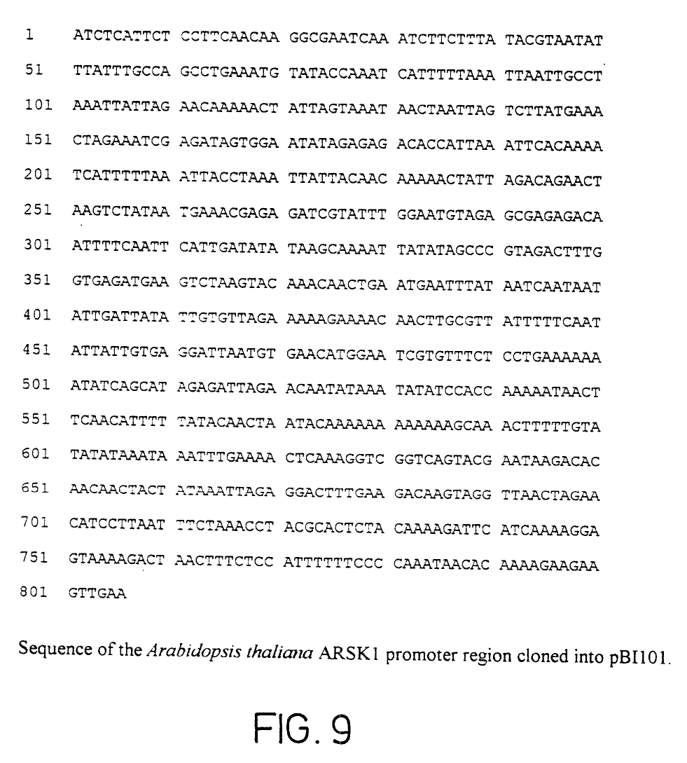

FIG. 9: shows the sequence of the A. thaliana ARSK1 promoter region cloned into pBI101 ( in Example 7).

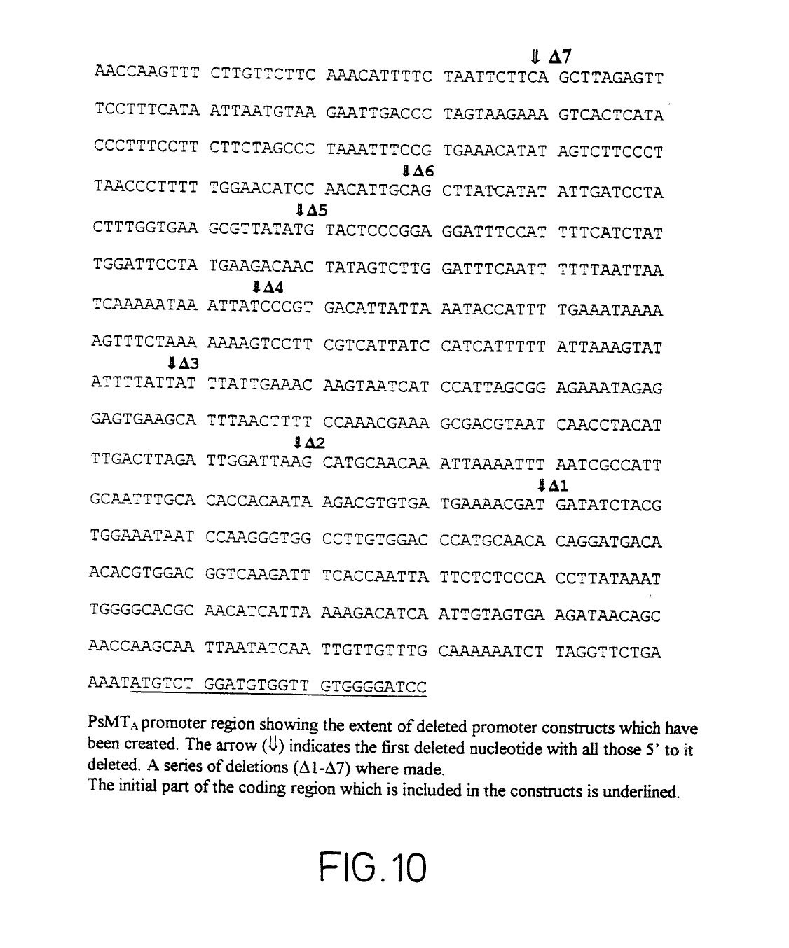

FIG. 10: shows the sequence of the PsMT_{A} promoter region, with the extent of the deleted promoter constructs which have been created.

### EXAMPLE 1: CLONING OF THE TUB-1 PROMOTOR

### DNA Preparation and Manipulation

Plasmid DNA was prepared from E. coli and Agrobacterium cultures by the alkaline lysis method (Sambrook et al, Molecular Cloning-A Laboratory Manual, Cold Spring Harbor Laboratory, N.Y. (1989)). Plasmid DNA was introduced into E. coli cells using the CaCl₂ transformation procedure (Sambrook et al, (1989) supra). Restriction digests and ligation reactions were carried out using the recommendations of the enzyme manufacturers.

DNA fragments were recovered from agarose gels using an electroelution chamber (IBI) according to the manufacturer's protocol. Oligonucleotides were synthesised on an Applied Biosystems 381A instrument and DNA sequencing of double-stranded plasmid DNA was carried out using an ABI automated sequencer according to. the manufacturer's recommendations.

### Cloning of the TUB-1 Promoter

Genomic DNA was prepared from Arabidopsis thaliana according to the method of Dellaporta et al, Plant Mol. Biol. Rep. 1: 19 (1983). The TUB-1 promoter region was amplified by PCR from the Arabidopsis genomic DNA using two oligonucleotide primers with the sequences:

5′ ATATTAAGCTTGTTACTGTATTCATTACGC 3′
and
5′ ACTATGGATCCGATCGATGAAGATTTTGGG 3′

designed from the published sequence of the TUB-1 upstream region (Oppenheimer et al, (1988) infra). Restriction enzyme sites HindIII and BamHI were incorporated into the primers to aid cloning of the amplified product. The PCR reaction comprised 7.5 ng genomic DNA, 200 μM dNTPs, 50 pmols of each primer and SuperTaq reaction buffer and enzyme at the concentration recommended by the manufacturer (HT Biotechnology Ltd.). 30 cycles of the amplification reaction were carried out with an annealing temperature of 55° C. and a 1 minute extension at 72° C.

The amplified DNA was digested with HindIII and BamHI and a specific DNA fragment of 560 bp was recovered from a 1% agarose gel by electroelution. This was cloned into the plasmid vector pUC19 (Yanisch-Perron et al, Gene, 33:103 (1985)) and the sequence of the TUB-1 promoter was verified.

The TUB-1 promoter, the sequence of which is shown in FIG. 1, was then introduced into the vector pBI101 (Clontech) as a HindIII/BamHI fragment. The HindIII and BamHI restriction sites introduced with the PCR primers are included in the sequence shown in FIG. 1. This vector contains the coding region of β-glucuronidase allowing the production of GUS to be used as a reporter of promoter activity in a transformed plant.

### Production of Transgenic Tomato Hairy Roots

pBI101 containing the TUB-1 promoter fragment was introduced into Agrobacterium rhizogenes strain LBA9402 by electrotransformation according to the method of Wen-jun & Forde, Nucleic Acids Research, 17:8385 (1989)). The bacteria were used to transform Lycopersicon esculentum cv. Ailsa Craig by a standard protocol (Tepfer, Cell, 37:959-967 (1984)).

Transgenic roots were cultured on 0.5 x Murashige and Skoog basal salts mixture supplemented with Gamborgs 5 vitamins, 3% sucrose (w/v) and 0.2% phytagel (w/v). 100 mgl-1 kanamycin was included during initial selection. Transgenic root lines were tested for the production of GUS by staining with X-gluc at a concentration of 1 mgml-1 in 100 mM phosphate buffer pH7.0 containing 10 mM EDTA , 0.1% (v/v) Triton X-100 and 0.5 M each of potassium ferricyanide and potassium ferrocyanide (Jefferson et al, (1987) supra; Schrammeijer et al, Plant Cell Reports 9: 55-60 (1990)). Root sections were incubated in the substrate for 12-16 hours.

### Infection of Roots with Globodera pallida and Meloidogyne incognita

The J2 of Globodera pallida were obtained from cysts and sterilised extensively before use. The cysts were soaked in running tap water for 2-3 days followed by an overnight soak in 0.1% malachite green at room temperature. Cysts were then rinsed for 8 h in running tap water prior to soaking overnight at 4° C. in an antibiotic cocktail (8 mg ml-1 streptomycin sulphate, 6 mg ml-1 penicillin G, 6.13 mg ml-1 polymicin B, 5 mg ml-1 tetracycline and 1 mg ml-1 amphotericin B).

The cysts were then washed in filter sterilised tap water and set to hatch in filter-sterilised potato root diffusate. The cysts were placed on a 30 μm nylon mesh secured over a plastic ring and contained within a jar containing a small amount of the sterile potato root diffusate. The jar was placed at 20° C. in the dark. The overnight hatch of J2s was collected and sterilised sequentially for 10 min each with the following antibiotics; 0.1% streptomycin sulphate, 0.1% penicillin G, 0.1% amphotericin B and 0.1% cetyltrimethyl-ammoniumbromide (Cetavlon). The nematodes were pelleted between treatments by brief (10 s) microcentrifugation. Following sterilisation, they were washed extensively in filter sterilised tap water prior to use.

Roots of transformed lines were cultured for 4 weeks before 2 cm lengths including root tips were transferred to fresh media. After a further 3-4 days, a 5-10 μl aliquot containing approximately 35 G. pallida J2 was pipetted onto each actively growing root approximately 1 cm from its tip. A 1 cm² piece of sterile GFA filter paper was placed over each inoculated area to aid infection and was removed 24 h later.

Infective juveniles of Meloidogyne incognita were obtained from egg masses taken from the galls of infected tomato roots. The galled roots were harvested and rinsed in tap water to remove excess soil. Egg masses were removed from the roots by hand using a scalpel and sterilised sequentially with 0.1% Penicillin G, 0.1% streptomycin sulphate and 0.1% amphotericin B for 30 min each followed by 5 min in 0.1% Cetavlon. The egg masses were then washed 5-6 times in sterile tap water before being placed on a 30 μm nylon mesh supported between two plastic rings in a jar containing approximately 5 ml of sterile tap water. Hatching occurred at 25° C. in the dark. The overnight hatch of juveniles was sterilised as for G. pallida and the transgenic roots infected in an identical manner.

### Investigation of TUB-1 Promoter Activity in Nematode Infected Transgenic Roots

At 7 day time intervals after infection sections were removed from infected transgenic hairy roots. Equivalent pieces were also removed from non-infected, control roots. The roots were rinsed briefly in distilled water to remove any adhering pieces of agar and then immersed in X-gluc solution as previously described. After overnight staining the roots were placed in 1% (v/v) sodium hypochlorite solution for 2 min then rinsed in water and plunged into boiling acid fuchsin (0.035% (w/v) in 25% (v/v) glacial acetic acid) for 2 min to stain the nematodes. Roots were then immediately rinsed in distilled water and incubated at 65° C. overnight in acidified glycerol to clear the root tissue.

Stained whole root segments were examined using a light microscope at low magnification (x4-x25) and infected areas were excised and sectioned to a thickness of 100 μm using a vibrating microtome (Oxford). Sections were then mounted in glycerol and examined under both light- and dark-field using a microscope (Leica DM).

### RESULTS

### Production of Transgenic Hairy Roots

A number of transgenic roots lines were obtained which became blue upon incubation with X-gluc. Two most consistently highly expressing lines were chosen for the infection experiments.

FIG. 2 shows the results of GUS expression under the control of the T-UB-1 promoter in transgenic hairy roots of tomato.

All roots were stained for GUS activity with X-gluc. In FIG. a), roots infected with Meliodogyne incognita show strong GUS expression in galls, 14 days after infection.

In b), strong expression of GUS in a large gall induced by M. incognita is shown 28 days after infection.

In c) can be seen a section through a gall caused by M. incognita; the centre of the gall stains intensely for GUS activity. In FIG. c), f =nematode feeding cells with particularly high TUB-1 promoter activity.

### Effect of Nematode Infection on TUB-1 Promoter Activity

Stained non-infected control roots were examined and it was clear that the most intense staining occurred in the root tips and at the sites of initiation of lateral roots. However, staining wets apparent along the whole length of the roots.

Roots infected with M. incognita showed a similar pattern of staining to uninfected roots. TUB-1 promoter was not down-regulated by nematode invasion. In addition, galled regions were stained more intensely than surrounding regions of root. These galled regions were then sectioned using a vibrating microtome to investigate the expression of the GUS gene within the gall. It was found that GUS was present throughout the section and the staining was particularly intense in the giant cells which make up the root-knot nematode feeding site. This heightened intensity at the site of nematode establishment may reflect the multinucieaze nature and high metabolic activity of these cells or it may represent a relative upregulation of the TUB-1 promoter in giant cells.

Roots infected with G. pallida had a large amount of necrotic tissue surrounding the sites of infection. These cells were presumably killed by the intracellular migration process and consequently they did not stain intensely. However, undamaged cells continued to express GUS. Sectioning of infected regions showed there to be GUS expression within the syncytium (cyst nematode feeding cell).

### EXAMPLE 2: CLONING OF PsMT_{A}

### DNA Preparation and Manipulation

As for Example 1.

### GUS Expression Directed by the PsMT_{A} Promoter

A DNA fragment containing 816 bp of 5′ flanking region and the first 7 amino acids of the coding sequence of PsMT_{A} was amplified by PCR and introduced as a HindIII/BamHI fragment into the vector pBI101.2 (Clontech). The sequence of this region is shown in FIG. 3. This resulted in a translational fusion between PsMT_{A} and GUS.

The construct was introduced into Agrobacterium tumefaciens LBA4404 by electroporation as for TUB-1. This strain was then used to transform Arabidopsis thaliana C24 according to the method of Clarke et al, Plant Molecular Biology Reporter, 10:178-189 (1992)).

Transformed Arabidopsis was grown on 0.5 x Murashige & Skoog media containing 10% sucrose (w/v) and 0.2% phytagel (w/v) and selected with 25 mgl-1 kanamycin. Staining of roots with X-gluc was then carried out as for TUB-1 transformed hairy roots.

Infective juveniles of M. incognita were prepared as before and inoculated onto root tips of transformed Arabidopsis seedlings which were 2-3 weeks old. Approximately 30 juveniles suspended in 2% w/v methyl cellulose were pipetted onto each selected root tip. At 7 day intervals after infection plants were carefully removed from the agar and the root systems rinsed in distilled water prior to staining with X-gluc as described previously. If necessary to visualise the nematodes the roots were then counter-stained with acid fuchsin. Roots were first soaked in 1% sodium hypochlorite for 30s then rinsed well in distilled water prior to immersion in boiling acid fuchsin stain (see Example 1) for 30 s. Root tissue was cleared in acidified glycerol as for Example 1.

### RESULTS

FIG. 4 shows the results of transgenic Arabidopsis roots expressing GUS under control of the PsMT_{A} promoter.

All roots were stained for GUS activity with X-gluc. In a) , uninfected roots showed strong expression of GUS throughout the root system.

In b), the root system of a plant infected with M. incognita 7 days after infection is shown. The arrow indicates a developing gall.

Uninfected roots of Arabidopsis plants transformed with PsMT_{A} promoter:GUS construct showed expression in the root system with slightly reduced staining in young, lateral root tips. Some expression was also observed in senescing aerial tissue. Plants infected with M. incognita still exhibited strong expression throughout the root system with more intense staining of gall tissue surrounding the nematode.

Infective juveniles of Heterodera schachtii were obtained from cysts and sterilised extensively before use. Cysts were incubated in 0.1 % malachite green for 30 minutes at room temperature and rinsed in running tap water for 1 h prior to soaking overnight at 4° C. in an antibiotic cocktail containing 8 mg ml⁻¹ streptomycin sulphate, 6 mg ml⁻¹ penicillin G, 6.13 mg ml⁻¹ polymyxin B, 5 mg ml⁻¹ tetracycline and 1 mg ml-⁻¹ amphotericin B. The cysts were washed and set to hatch in filter-sterilised tap water. An overnight hatch of J2s was counted and sterilised sequentially for 5 min periods with each of the following antibiotics; 0.1% streptomycin sulphate, 0.1% penicillin G, 0.1% amphotericin B and 0.1% cetyltrimethylammoniumbromide; Cetrimide (Sigma Chemical Co., Dorset, U.K.). J2s were collected by microcentrifugation for 10 seconds between treatments and were finally washed extensively in filter sterilised tap water before use.

Sterilised juveniles were inoculated onto root tips of transformed Arabidopsis seedlings as described for M. incognita supra. Plants were removed from the agar at 2 day intervals until 14 days after infection and then at 21 and 28 days after infection. Root systems were stained and examined as for infections with M. incognita(supra).

### RESULTS

Arabidopsis plants transformed with the PsMT_{A} promoter:GUS construct and infected with H. schachtii exhibited strong expression throughout the root system and around the site of infection of the nematode until 14 days after infection. FIG. 5 shows the results of A. thaliana transformed with PsMT_{A}:GUS construct and infected with Heterodera schachtii. The A. thaliana were stained for GUS activity at: A) 2 days post infection; B) 6 -days post infection; C) 6 days post infection and D) 8 days post infection. The nematode is indicated with an arrow in each case. (See FIG. 5). By 21 days after infection there was some localised down-regulation of the promoter around the site of nematode infection.

### EXAMPLE 3: EXPRESSION OF THE ENGINEERED ORYZACYSTATIN (OC1ΔD86) REGULATED BY THE TUB-1 PROMOTER

### DNA Preparation and Manipulation: as for Example 1.

The GUS gene was removed from the commercially available plasmid PBI121 (Clontech) as a BamHI-SstI fragment. A synthetic oligonucleotide linker was ligated into the cut vector such that the BamHI and SstI sites were recreated, and an additional KpnI site was introduced between them.

The resulting plasmid was digested with HindIII and BamHi to remove the CaMV35S promoter which was directly replaced by the TUB-1 promoter, also as a HindIII-BanHI fragment. The coding region of the engineered oryzacystatin gene (OC1ΔD86) was inserted into the plasmid behind the TUB-1 promoter as a BamHI-KpnI fragment.

The final construct was introduced into Agrobacterium tumefaciens LBA4404 by electroporation, as in Example 2. The plasmid-containing bacteria were used to transform Arabidopsis thaliana C24, as in Example 2.

### EXAMPLE 4: EXTENDING TUB-1 PROMOTER SEQUENCES

The 560 bp fragment of the TUB-1 promoter which was used to make the TUB-1:GUS construct described in Example 1 was identified as too short to confer suitable expression in transgenic Arabidopsis (Leu et al., The Plant Cell, 7:2187-2196 (1995) and our own observations). However, the fact that it was capable of directing GUS expression in transgenic tomato hairy roots and transgenic potato shows that the 560 bp TUB-1 promoter fragment is useful in some crop species. An inverse PCR technique was used to clone longer fragments of the TUB-1 promoter for use in other crop plants to provide root-specific expression.

### Method for Obtaining Extended TUB-1 Promoter Sequences

1 μg of Arabidopsis thaliana C24 DNA, prepared as described in Example 1, was digested with BAMHI and the reaction mix extracted with phenol/chloroform and precipitated with ethanol following the addition of 0.1 volumes 3 M sodium acetate pH 4.8. The precipitated DNA was self-ligated overnight at 16° C. and the ligation reaction was then used as a template for PCR. The primers used in the amplification were:

5′ CGTAATGAATACAGTAACTTTGC 3′
and
5′ CAAGAACTCATCCTACTTTGTTG 3′

Reaction conditions for PCR were as described in Example 1. Electrophoresis of the PCR products on an agarose gel revealed a single DNA band of 400 bp which was isolated from the gel by electroelution and cloned into the pCRII vector (Invitrogen). The DNA insert was completely sequenced on both strands and this enabled the design of a further oligonucleotide primer which could be used with an existing primer to amplify a longer region of the TUB-1 promoter consisting of approx. 920 bp of upstream sequence. The sequence of this primer, designated TUB900 was:

5′ ACAAAGCTTTACAAGTTCAATTATTG 3′

It was used in conjunction with the primer previously described in Example 1:

5′ ACTATGGATCCGATCGATGAAGATTTTGGG 3′

in a PCR reaction comprising 7.5 ng Arabidopsis genomic DNA as described previously in Example 1. The PCR products were digested with Bam HI and HindIII, electrophoresed through an agarose gel, purified by electroelution and cloned into the plasmid vector pUC19 as described previously. The DNA insert was sequenced and confirmed as an extended fragment of the TUB-1 promoter (see FIG. 6). The approximately 900 bp fragment was then cloned into the vector pBI101 as before. The approach can be used to extend the known sequence of the TUB-1 upstream region even further if a longer promoter fragment proves necessary for any crop species. The approach can be used to isolate promoter regions of any gene providing root-specific expression if unknown additional upstream sequence is needed to ensure the specific pattern of expression required.

### EXAMPLE 5: CONSTRUCT OF THE TUB-1 PROMOTER AND THE ANTI-NEMATODE PROTEIN MODIFIED ORYZACYSTATIN

In this example, the 560 bp TUB-1 promoter fragment, from Example 1 was cloned into a plant transformation vector in conjunction with a modified plant cysteine proteinase inhibitor (cystatin). This work was carried out to demonstrate that the promoter can deliver biologically active expression levels of an anti-nematode protein using a cystatin as a specific example.

### DNA Preparation and Manipulation

As for Example 1.

Preparation of the TUB-1:OcIΔD86 construct

The commercially available plasmid pBI121 (Clontech) consists of the GUS gene under the control of the CaMV35S promoter. The GUS gene was removed from this plasmid as a BamHI-Sst I fragment and replaced with a synthetic oligonucleotide linker which recreated the BamHI and SstI sites and introduced an additional KpnI site between them.

The resulting plasmid was digested with HindIII and BamHI to remove the CaMV35S promoter and this was directly replaced by the TUB-1 promoter, also as a HindIII-BamHI fragment. The oryzacystatin gene, Oc-I, has been modified to produce a variant (Oc-IΔD86) which has a greater detrimental effect on the growth and development of nematodes (Urwin et al., The Plant Journal, 8:121-131 (1995)). This modified gene was cloned as a BamHI-KpnI fragment into the plant transformation vector containing the TUB-1 promoter.

The resulting construct was introduced into Agrobacterium tumefaciens strain LBA4404 by electroporation as described for Example 1. The construct was introduced into potato according to the method of Dale & Hampson (Euphytica, 85:101-108 (1995)) and initial analysis of the Oc-IΔD86 content of leaf and root tissue has been carried out for a number of plants.

### Determination of Oc-IΔD86 Levels in Transgenic Potato Plants

Samples of potato root or leaf tissue were ground to a fine powder in liquid nitrogen and resuspended in PBS buffer supplemented with 2.5 μM trans-Epoxysuccinyl-L-Leucylamido (4-Guanido)-butane (E64) at levels that were somewhat more than required to inhibit native proteinases without sufficient excess to bind to all papain in the plate wells in the later assay. This level is found empirically for different plant tissues by increasing E64 concentrations in preliminary ELISA assays until further addition does not enhance detection of added Oc-IΔD86 in the range 0-1% total soluble protein (tsp). Aliquots of protein extract were added to the wells of a microtitre plate previously coated with papain (10 μg/well) to capture the Oc-IΔD86. This was then quantified by a standard two-antibody sandwich ELISA (Harlow & Lane, Antibodies-A laboratory manual, Cold Spring Harbor, N.Y. (1988)) using a polyclonal antibody raised against Oc-I and an alkaline phosphatase conjugated rabbit anti-rat secondary antibody diluted 1 in 2,000. Alkaline phosphatase activity was measured by monitoring p-nitrophenyl phophate hydrolysis at 405 nm. Non-transformed potato extract spiked with purified recombinant Oc-IΔD86 (0-1% tsp) was used to construct a standard curve. Potato plants transformed with a CaMV35S:Oc-IΔD86 construct were analysed in the same way for comparison.

### RESULTS

As expected, the constitutive promoter CaMV35S directed expression of Oc-IΔD86 in both leaf and root tissue of transformed potato plants. In contrast, the TUB-1 promoter provided similar expression levels in roots but no detectable level in leaves (see Table). In all cases, values were compared with values for the corresponding tissue of untransformed potato plants. The expression of an anti-nematode protein, in this case a proteinase inhibitor, can therefore be restricted to root systems.

| Construct | Leaf | Root |
|---|---|---|
| promoter/effector | (% tsp) | (% tsp) |
| CaMV35S:Oc-IΔD86 | 0.058 ± 0.003** | 0.096 ± 0.009*** |
| TUB-1:Oc-IΔD86 | 0 ± 0.0007 NS | 0.077 ± 0.003** |

Table 1. Estimated expression levels as % of total soluble protein (% tsp) in leaf and root tissue of transformed potato plants for the effector protein Oc-IΔD86 given by two constructs differing only in promoters. Values are for example lines and estimates were provided by ELISA (see text for details). Values were compared using One-way ANOVA with a priori contrasts against corresponding untransformed tissue (NS, not significant P=0.5; **, P<0.01; ***, P<0.001).

The RPL16A gene from Arabidopsis thaliana encodes the ribosomal protein, L16. Transcription of the RPL16A promoter is cell specific and promoter:GUS fusions show it to be expressed in internal cell layers behind the root meristem, dividing pericycle cells of mature roots, lateral root primordia and the stele of developing lateral roots. Expression was also observed in developing anthers and pollen (Williams & Sussex, The Plant Journal, 8:65-76(1995)).

The ARSK1 gene from Arabidopsis thaliana encodes a protein with structural similarities to serine/threonine kinases. Its expression is root specific as judged from a promoter:GUS fusion construct reintroduced into Arabidopsis. There were high levels of expression in the epidermal, endoepidermal and cortex regions of the root (Hwang & Goodman, The Plant Journal, 8:37-43 (1995)).

### EXAMPLE 6: CLONING OF THE RPL16A PROMOTER

### DNA Preparation and Manipulation

As for Example 1.

### GUS Expression Directed by the RPL16A Promoter

Genomic DNA was prepared from Arabidopsis thaliana as for Example 1. The RPL16A promoter region was amplified by PCR from the Arabidopsis genomic DNA using two oligonucleotide primers with the sequences:

5′ ACAAAGCTTAACGAAAGCCATGTAATTTCTG 3′
and
5′ ACAGGATCCCTTCAAATCCCTATTCACATTAC 3′

designed from the published sequence of the RPL16A upstream region (Williams & Sussex, The Plant Journal, 8: 65-76 (1995)). Restriction enzyme sites HindIII and BamHI were incorporated into the primers to aid cloning of the amplified product. PCR amplification of the RPL16A promoter fragment was carried out as described in Example 1. The amplified DNA was digested with HindIII and BamHI and a specific DNA fragment was recovered from an agarose gel and cloned into the plasmid vector pUC19 (Yanisch-Perron et al., (1985) infra). The sequence of the RPL16A promoter was verified (see FIG. 7).

The RPL16A promoter was then introduced into the vector pBI101 (Clontech) as a HindIII/BamHI fragment.

Introduction of the construct into Agrobacterium tumefaciens LBA4404 and transformation of Arabidopsis thaliana with the RPL16A:GUS construct was as described for Example 2. Staining of roots with X-gluc was carried out as described for TUB-1 transformed hairy roots.

### RESULTS

Uninfected roots of Arabidopsis plants transformed with the RPL16A promoter:GUS construct showed expression particularly in lateral root primordia and internal cell layers just behind the root tip. FIG. 8 shows the results of A. thaliana transformed with the RPL16A:GUS construct and stained for GUS activity. In the Figure A) GUS expression is evident in cells behind the root meristem and in developing vascular tissue and B) GUS expression occurs in a lateral root primordium.

### EXAMPLE 7: CLONING OF THE ARSK1 PROMOTER

### DNA Preparation and Manipulation

As for Example 1.

GUS Expression Directed by the ARSK1 Promoter

A DNA fragment containing a region of the ARSK1 promoter was amplified from Arabidopsis thaliana genomic DNA by PCR as described in Example 1 using two oligonucleotide primers with the sequences:

5′ ACAAAGCTTATCTCATTCTCCTTCAAC 3′
and
5′ ACAGGATCCTTCAACTTCTTCTTTTG 3′

designed from the published sequence of the ARSK1 upstream region (Hwang & Goodman, The Plant Journal, 8:37-43 (1995) and GenBank Accession No. L22302).

The amplified DNA fragment was digested with HindIII and BamHI and cloned into the plasmid vector pUC19 as described in Example 1. The ARSK1 promoter was then introduced into the vector pBI101 (Clontech) as a HindIII/BamHI fragment (sequence shown in FIG. 9). The construct was introduced into Agrobacterium tumefaciens LBA4404 by electroporation as for TUB-1 and this was then used to transform Arabidopsis thaliana C24 as described in Example 2.

### EXAMPLE 8: MANIPULATION OF PROMOTER REGIONS TO ENHANCE SPECIFICITY

This example describes how promoter deletions may be created to identify regions of the promoter which are essential for expression in roots and/or to manipulate a promoter to have greater root specificity. This example uses the promoter from the pea metallothionein-like gene, PsMT_{A}.

### NA Preparation and Manipulation

As for Example 1.

### Preparation of Deletion Constructs

A total of 7 deletion constructs were created in the vector pBI101.2, designated PsMT_{A} Δ1 (210 bp), PsMT_{A}Δ2 (282 bp), PsMT_{A}Δ3 (393 bp) , PsMT_{A}Δ4 (490 bp), PsMT_{A}Δ5 585 bp) PsMT_{A}Δ6 (632 bp) and PsMT_{A}Δ7 (764 bp),

For Δ1, Δ2, Δ5, Δ6, and Δ7 restriction sites were used to create the deletions, which were subcloned into pUC18 and then transferred to pBI101.2 as Hind III/Bam HI fragments. The extent of the deletions and the restriction sites used are indicated on FIG. 10.

For the Δ3 and Δ4 constructs no suitable restriction sites were available so oligonucleotide primers were synthesized and used in PCR reactions to amplify the desired promoter regions. The primers for the Δ3 deletion were:

| | |
|---|---|
| 5′ ATTTATTGAAACAAGTAATCATCC 3′ | |
| and | |
| 5′ GGAAACAGCTATGACCATG 3′ | (M13 reverse |
| | primer). |

The primers for the Δ4 deletion were:

| | |
|---|---|
| 5′ TATTAAGCTTCCCGTGACATTATTAAATAC 3′ | |
| and | |
| 5′ GGAAACAGCTATGACCATG 3′ | (M13 reverse |
| | primer) |

The template for the PCR reaction in each case was a pUC18 plasmid clone containing the complete PsMT_{A} promoter region as a Hind III/Bam HI fragment. Conditions for the PCR reaction were as described in Example 1. The amplified fragment from the Δ3 PCR was cloned directly into pCRII (Invitrogen) and verified by sequencing. A Hind III/Bam HI fragment containing the deleted promoter was then cloned into pBI101.2.

The product of the Δ4 PCR was digested with Hind III/Bam HI, cloned first into pUC18, and from there into pBI101.2.

Constructs were introduced into Agrobacterium tumefaciens as in Example 1 and have been used to transform Arabidopsis.

### RESULTS

Transformants have been recovered for the Δ2, Δ5 and Δ6 deletion reporter constructs. When stained with X-gluc to reveal GUS activity as described in Example 1, the Δ5 and Δ6 plants showed an identical pattern of expression to plants transformed with the full length promoter construct. In contrast, plants transformed with the Δ2 construct displayed no GUS activity in roots but only in leaf hydathodes, and some flower parts. This implies that a region between −585 and −282 bp must be responsible for expression in root tissue. The Δ3 and Δ4 constructs should define more precisely the role of this region of DNA and it may then be possible to use this information to create a promoter construct which has only activity in roots.

1. A nucleic acid molecule comprising;
i) a promoter characterised in that it is capable of directing expression predominantly in the roots of a plant, and naturally controls expression of a gene that does not respond to pathogenic infection of said plant;
ii) and a sequence which encodes an anti-nematode protein.

2. A nucleic acid molecule according to claim 1 wherein the promoter is the promoter from the b1-tubulin gene (TUB-1), or the promoter from the metallothionein-like gene (PsMT_{A}), or the promoter from the ribosomal protein gene (RPL16A), or the ARSK1 gene promoter.

3. A nucleic acid molecule according to claim 1 or 2 wherein the promoter is the promoter from the b1-tubulin gene of Arabidopsis.

4. A nucleic acid molecule according to claims 1 or 2 wherein the promoter is the promoter from the metallothionein-like gene of Pisum sadvum.

5. A nucleic acid molecule according to claims 1 or 2 wherein the promoter is the promoter from the ribosomal protein gene RPL16A of Arabidopsis thaliana.

6. A nucleic acid molecule according to claims 1 or 2 wherein the promoter is the promoter from the ARSK 1 gene of Arabidopsis thaliana.

7. A nucleic acid molecule according to claims 1 to 6 which also comprises a transcription termination sequence.

8. A nucleic acid molecule according to claims 1 to 7 wherein the anti-nematode protein is effective against one or more of the following nematode genera, Heterodera, Globodera, Meloidogyne, Hoplolaimus, Helicotylenchus, Rotylenchoides, Belonolaimus, Paratylenchus, Paratylenchoides, Radopholus, Hirschmanniella, Naccobus, Rotylenchulus, Tylenchulus, Hemicycliophora, Criconemoides, Circonema, Paratylenchus, Trichodorus, Paratrichodorus, Longidorus, Paralongidorus or Xiphinema.

9. A nucleic acid molecule according to claim 8 wherein the anti-nematode protein is effective against one or more of the following nematodes, Meloidogyne incognita, M. javanica, Globodera pallida, G. rostochiensis, Heterodera schachtii, Heterodera glycines, M. arenariaor M. hapla.

10. A nucleic acid molecule according to claim 1 to 9 wherein the anti-nematode protein is a collagenase, a lectin, an antibody, a toxin of Bacillus thuringiensis or a proteinase inhibitor.

11. A nucleic acid molecule according to claim 10 wherein the protein is a cystatin.

12. A nucleic acid molecule according to claim 11 wherein the cystatin is oryzacystatin 1, having the amino acid 86 deleted (or OC1ΔD86).

13. A nucleic acid molecule according to claim 1 to 12 which is in the form of a vector.

14. The use of a nucleic acid molecule comprising a promoter that is capable of directing expression predominantly in the roots of a plant, and naturally controls expression of a gene that does not respond to pathogenic infection of said plant in the preparation of a nucleic acid construct adapted to express an anti-nematode protein.

15. The use according to claim 14 modified by any one or more of the features of any one of claims 2 to 13.

16. A method of preparing a nucleic acid molecule as defined in any one of claims 1 to 12 which comprises coupling together successive nucleotides, and/or ligating oligo-and/or poly-nucleotides.

17. A method according to claim 16 wherein the nucleic acid molecule is prepared recombinantly.

18. A method of conferring nematode resistance on a plant which comprises the step of transforming the plant with a nucleic acid molecule as defined in any one of claims 1 to 12.

19. The use of a nucleic acid molecule as defined in any one or claims 1 to 12 in the preparation of a transgenic plant having nematode resistance.

20. A plant cell transformed with a nucleic acid molecule as defined in any one of claims 1 to 12.

21. A plant comprising plant cells according to claim 20.

22. A process for the manipulation of a promoter to alter the specificity of same.
